(19) United States
(12) Patent Application Publication
(10) Pub. No.: US 2010/0330662 A1
(43) Pub. Date: Dec. 30, 2010

(54) Apparatus, System and Method for Consumer Detection of Contaminants in Food Stuffs

(75) Inventors: Morton Greene, Potomac Falls, VA (US); Edward Yokley, Anderson, SC (US)

Correspondence Address: EDWARD F. BEHM;DRINKER BIDDLE & REATH LLP, Ste 2000, One Logan Square, Philadelphia, PA, 19103-6996, US

(73) Assignee: Inkode Corporation

(21) Appl. No.: 12/875,513

(22) Filed: Sep. 3, 2010

Related U.S. Application Data
(63) Continuation-in-part of application No. 12/633,129, filed on Dec. 8, 2009.
(60) Provisional application No. 61/201,294, filed on Dec. 8, 2008.
(60) Provisional application No. 61/209,236, filed on Mar. 4, 2009.

(51) Int. Cl. C12M 1/34

(52) U.S. Cl. 435/287.9

(57) ABSTRACT

An apparatus, system and method of detecting contaminants, such as salmonella, in at least one ingestible item. The apparatus, system and method may include a disposable detector having therein at least one circuit layer, wherein a reaction of the reactant with at least a portion of the at least one circuit layer indicates, to the consumer user, a presence of a contaminant.

### CROSS-REFERENCE TO RELATED APPLICATIONS

This application is a continuation-in-part of U.S. patent application Ser. No. 12/633,129, filed Dec. 8, 2009, entitled “Apparatus, System And Method For Consumer Detection Of Contaminants In Food Stuffs”, which application claims priority to U.S. Provisional Application No. 61/201,294, filed Dec. 8, 2008, entitled “Detection Of Melamine Fluorescent Derivatives”, and U.S. Provisional Application No. 61/209,236, filed Mar. 4, 2009, entitled “Apparatus, System And Method For Consumer Detection Of Contaminants In Food Stuffs”, the entire disclosures of which are incorporated by reference herein as if each set forth herein in their entirety.

### BACKGROUND OF THE INVENTION

1. Field of the Invention

The present invention is directed to chemical testing, and, more specifically, to an apparatus, system and method for consumer detection of contaminants in food stuffs.

2. Description of the Background

Melamine is a symmetrical triaminotriazine and a known item of industrial chemical commerce. Illustrated in FIG. 1, melamine is commonly used in the manufacture of waterborne resins that are crosslinked to form Melamine-formaldehyde binder resins for, for example, countertop and flooring laminates, adhesives, dinnerware and many other products.

Melamine has also been investigated as a nitrogen source for plants, and agricultural animals such as cattle. In general, such fertilizer and dietary applications have not been successful due to the relatively slow breakdown of melamine in plants and animals. Due to the low water solubility, precipitation from biological fluids has been observed, leading to kidney stones and renal toxicity in humans.

There has recently been an unfortunate use of melamine by unscrupulous suppliers of animal feeds and human baby formula products as an additive in their products to raise the analyzed Nitrogen content, and thereby the implied protein content, of these food products. Responsive to these unscrupulous inclusions of melamine, toxic kidney reactions have been observed, leading to deaths in both pediatric and veterinary practice.

However, the ability to test for the presence of melamine has, to date, been limited. For the most part, current melamine testing is forensic or industrial in nature, and consequently most melamine testing apparatuses consist of large, inconvenient equipment that does not lend itself to testing outside of governmental and/or laboratory facilities.

There is a need therefore for a fast, simple, inexpensive, and most preferably hand held analytical device that is specific to the detection of Melamine and other contaminants at low levels, and that does not require the transportation of samples to external laboratories. To address this need, the present invention includes a detection device, system and method based on UV/fluorescence spectroscopy of derivatized melamines.

### SUMMARY OF THE INVENTION

The present invention includes an apparatus, system and method of detecting contaminants, such as Melamine, in at least one ingestible item. The apparatus, system and method may include a disposable notched probe having therein at least one send and one receive fiber optic, or electrical, or heat source, and a reactant associated with said disposable notched probe, wherein a reaction of the reactant with at least a portion of the ingestible item indicates, to the consumer user, a presence of a contaminant.

More specifically, melamine detection may include the steps of derivitizing the at least on ingestible item with an aromatic aldehyde, and detecting a spectroscopic variation indicative of Melamine presence in a reaction product of the derivitizing.

Thus, the present invention provides a fast, simple, inexpensive, and most preferably hand held analytical device that is specific to the detection of Melamine and other contaminants at low levels, and that does not require the transportation of samples to external laboratories.

### DESCRIPTION OF THE DRAWINGS

The present invention will be described in conjunction with the following figures, wherein like numerals denote like aspects of the invention, and wherein:

FIG. 1 illustrates a melamine chemical compound;

FIG. 2 illustrates a melamine reaction with aromatic aldehydes;

FIG. 3 illustrates exemplary aromatic aldehyde compounds;

FIG. 4 illustrates the resulting chemical compounds from the reaction of melamine with the aromatic aldehydes of FIG. 3;

FIG. 5 illustrates a melamine-phthalic anhydride condensation;

FIG. 6 illustrates a melamine reactive surface derivitization;

FIGS. 7A and 7B illustrate a fiber-optic probe; and

FIG. 8 illustrates a notched fiber-optic probe.

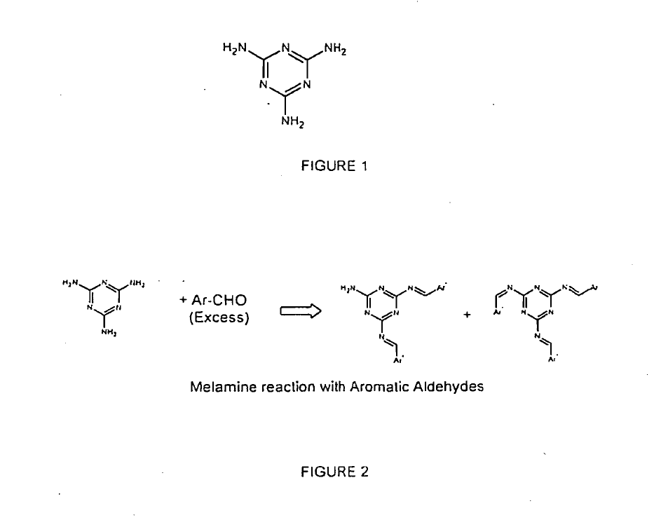

### DETAILED DESCRIPTION OF THE INVENTION

It is to be understood that the figures and descriptions of the present invention have been simplified to illustrate elements that are relevant for a clear understanding of the present invention, while eliminating, for the purposes of clarity, many other elements found in typical chemical detection apparatuses, systems and methods. Those of ordinary skill in the art will recognize that other elements are desirable and/or required in order to implement the present invention. However, because such elements are well known in the art, and because they do not facilitate a better understanding of the present invention, a discussion of such elements is not provided herein.

Melamine is known to react rapidly and cleanly with aldehydes. This rapid Mannich type reaction with formaldehyde is the basis for the wide application in thermoset resins. Thus the present invention includes a device based on the rapid derivitization of Melamine with aromatic aldehydes to give reaction products with extended conjugation and unique spectroscopic properties. In particular the di and tri condensation products are materials with extended conjugation that will offer differentiable spectroscopic properties with low degrees of interference from other aryl amine analyates. Examples of these reactions are discussed immediately hereinbelow.

A Melamine-Aromatic Aldehyde reaction, as illustrated in FIG. 2, yields the corresponding di and tri imines that, in particular, yield unique absorbtion/fluorescence spectra. These structures are sufficiently unique to isolate them spectroscopically for interferences with other pyridine and pyrimidene derivatives.

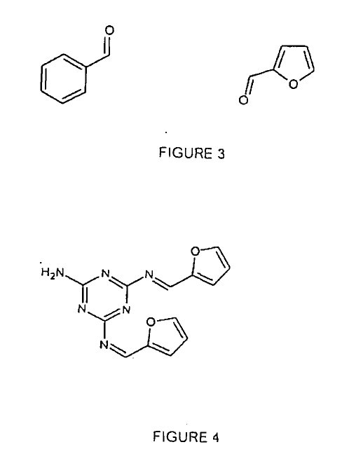

This reaction is particularly of interest in the case where the aromatic aldehyde is taken from the list of: benzaldehyde, substituted benzaldehydes, such as 2 and 4-nitrobenzaldehyde, 2 and 4-methylbenzaldehyde, 2 and 4-methoxy and alkoxybenzaldehydes. As shown in FIG. 3, the derivatives arising from reaction with furfural are also of interest in that the spectroscopic properties of these derivatives will be unique. Examples of the resulting structures are shown in FIG. 4.

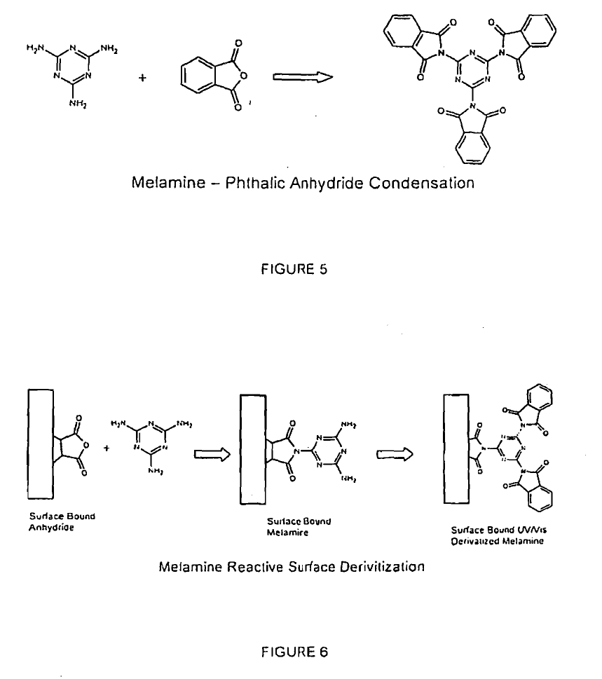

As another approach to unique Melamine derivatives, the known reaction of Melamine with cyclic aromatic anhydrides, such as phthalic anhydride, may be invoked. The product of this reaction and condensation produces a product with an extended ring structure and unique UV/Visible absorbance spectra. This condensation is shown in FIG. 5.

Also of interest are structures comprising anhydrides bound to surfaces, such as maleic or citraconic anhydrides copolymerized with other acrylic monomers to form a surface with pendant anhydride groups. On treatment with Melamine-containing samples, initial binding of one of the amino groups of the triazine to the surface is easily achieved. The subsequent reaction of the remaining groups with Phthalic Anhydride to yield a spectrscopically unique derivative bound to the surface leads to facile analysis of the surface coated cell. The stepwise reaction is illustrated in FIG. 6. In this case, the surface binding reaction is confined to one Melamine reactive site due to steric hindrance, leaving the remaining amines to be reacted with the anhydride.

Each of the aforementioned reactions, as discussed hereinabove, results in a detectable spectroscopic event. In preferred embodiments, such spectroscopic events would be detected using a portable, consumer-centric apparatus for detection even after purchase.

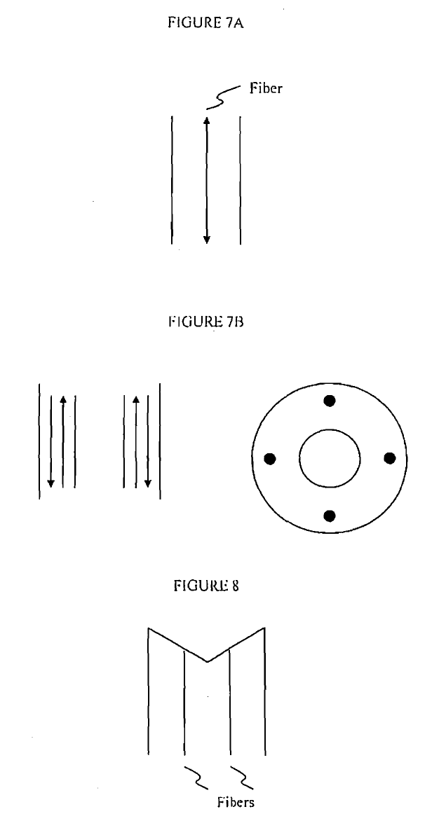

For example, FIGS. 7A and 7B illustrated exemplary, fiber-optic based probes for use in the present invention. The illustrated probes may include, for example, plastic-based probes, which may be similar to, for example, known glucose probes, threaded with fiber optics capable of detecting the aforementioned spectroscopic event. In order to create the reactive surface capable of generating the spectroscopic event, those skilled in the art will appreciate that the plastic of the probe may be doped with an agent that is reactive with melamine, such as, for example, tin or chrome.

As illustrated in FIG. 8, the fiber optics may be on opposing sides of the probe, such as in a send and receive arrangement, particularly in embodiments wherein a notch is placed in the probe, such as optically centered between the send and receive aspects of the probe. The probes may, for example, be sheathed, such as in an aluminum sheathing. The sheathing may be electrically, rather than optically, employed, such as by providing an ultrasonic, RF, AC, and/or DC sourcing, such as to test electrical properties of a subject under test. For example, an ultrasonic quartz, or similar electrical, model may enable the fluorescence of certain reactants, such as salmonella.

Further, for example, electrically, a wheatstone bridge type arrangement may allow for testing of conductivity/resistivity of, for example, liquids, such as milk, solids, such as using a probe. In such an embodiment, the wheatstone bridge may be coated with one or more coatings, wherein the coating(s) react with one or more undesirable materials, such as bacteria, such as salmonella, harsh chemicals, or the like, to change the resistivity of the coating(s), and to thereby detectably change the resistivity of the wheatstone bridge. Of course, one coating may effect different resistivity changes for different contaminants, or each coating may be uniquely affected by only one contaminant. In the latter embodiment, multiple wheatstone probe detectors may be used to each detect different contaminants. Thus, the presence of electrically detectable materials in a subject under test, such as dioxins, may be detected.

Likewise, heat may be provided to a subject under test, such as via the aforementioned electrical sourcing. Thereby, heat-activated reactants may be detected via the use of the present invention. Further, by providing heat, controlled reactants may be timed-released into a sample, such as wherein pellets are provided and melted by the heat source to perform a controlled release. Thus, the present invention may include reactant liquids or pellets for testing using the probe of the present invention. Additionally, the probe or reactants could be encapsulated, and, once the encapsulation seal is broken, friction can occur and the probe tip may be moistened with a reactant. As will be understood by those skilled in the art, various encapsulation methods may be employed, such as micro or nano encapsules, reactant dispensers, or multiple encapsules to provide multiple reactants, wherein the probe may be frictionally passed through the multiple reactants prior to, or during, use.

As discussed above, many tests require some period of incubation to raise the concentration of target reactants that may be detected by an antigen-antibody reaction. Fluorescent, stain based or other spectroscopic methods may require incubation periods of approximately 1-5 days depending on the test type administered. Such methods may be based on analysis of the bacterial cells themselves or of some byproduct of the metabolism of the cells. In this regard, previous approaches have required a slow culturing or growth step to generate sufficient target cells to analyze or to make enough metabolite to analyze.

However, reliance on incubation to achieve a detectable level of either cells or a unique metabolite that can be measured can be difficult and unreliable. An example of such an approach is the use of an optical fiber coated with an antibody coating to bind salmonella cells, which may be detected using an evanescent wave guided through the long path length of the optical fiber.

In an embodiment of the present invention, a method for the detection of the metabolic activity of the target reactant itself, such as salmonella cells, is provided. A detector of the present invention may include a film or coating medium that may have multiple functional components, including at least one of a selective immunologic binding component of the bacterial cells of interest; a cell culture fuel for the selectively attaching the bacteria; and an electrically conductive binder that is disconnected by the metabolic activity of the consumption of the culture fuel. In this way, the selection of only the target reactant of interest is may be achieved, with subsequent detection achieved by detecting the metabolic activity of the target reactant.

The timing and sensitivity of the detector may be regulated by the relative composition of the film or coating, the thickness and geometry of the film or coating, and/or the conductive sensitivity of the monitoring circuit. In an embodiment of the present invention, a monitoring circuit may monitor the change from conductive to not conductive as the integrity of the detection film may be degraded by the metabolic activity of the target reactant. The film may be designed to be specifically corroded by the target reactant, which may allow for a robust device design.

Target reactant specific corrosion may also allow for the placement of all necessary components on a single test strip. In such an embodiment, the conductivity monitoring and circuitry may be consolidated in an easily portable module. An electronic module may be used, for example, with a strip sensitized to specific immunologic binding of different bacterial pathogenic threats of interest that may be detected using the present invention.

Thus, the present detection apparatus and system may provide reaction detection, such as via chromatography, corrosion, and/or spectroscopy, to alert a user of the presence of a dangerous reactant, such as melamine. The present device may be a simplistic device, such as with inexpensive, disposable plastic probes as discussed hereinthroughout, and the testing in the present invention may thus be constituted simply by a review of peak intensity to assess the presence of threshold concentrations of particular reactants.

The present device may provide a readily understandable result for a non-scientific, consumer user. Thus, for example, the detector may simply detect a threshold concentration of a reactant in a subject under test, and accordingly provide simply a “YES” or “NO” answer as to the presence of the reactant under test. Needless to say, the threshold concentration sought is most preferably correspondent to a threshold level for a dangerous concentration of the reactant under test.

For example, the present invention may, with respect to melamine, detect concentrations as low as ½ part per million. However, the threshold sought with respect to melamine may be corresponded to that set forth by the Food and Drug Administration as constituting a dangerous melamine concentration, namely 2 parts per million. Thus, for any concentration of melamine higher than 2 parts per million, the present invention may provide a simple indication, such as a light, word, letter, symbol, or the like, to alert the user of the dangerous level of melamine in the subject under test.

Those skilled in the art will appreciate, in light of the disclosure hereinabove, that the aforementioned and various other detection methodologies and devices are suitable for use in the present invention. Such devices may detect the dispersion of the detected subject's light into component colors, energies, and wavelengths, and/or may detect modification of the physical properties of the detected subject by inference, such as through detection of temperature, mass, luminosity and/or composition, for example.

What is claimed is:

1. A system for detecting a target reactant in at least one ingestible item, comprising:
a wheatstone bridge detector having at least one coating, wherein the coating comprises at least one of a selective immunologic binding component of the target reactant and a fuel for the selectively attaching the target reactant, and at least one electrically conductive binder; and
a circuit monitor connected to the detector, wherein the at least one electrically conductive binder forms a closed circuit having a variable resistance response to a presence of the target reactant.

2. The system of claim 1, wherein the at least one coating comprises a homogeneous mixture.

3. The system of claim 1, wherein the fuel is an energy source selectively preferred by the target reactant.

4. The system of claim 3, wherein the closed circuit is broken by the consumption of the fuel.

5. The system of claim 1, wherein the detector is plastic-based.

6. The system of claim 1, wherein the target reactant comprises salmonella.

7. The system of claim 6, wherein an amount of the salmonella present in the ingestible item is at least about 0.5 parts per million.

8. The system of claim 6, wherein an amount of the salmonella present is at least about 2 parts per million.

9. The system of claim 1, wherein the at least one electrically conductive binder comprises aluminum.

10. The system of claim 1, wherein circuit monitor provides at least one of the group consisting of RF, AC and DC sourcing.
